(19) United States
(12) Patent Application Publication
(10) Pub. No.: US 2011/0117032 A1
(43) Pub. Date: May 19, 2011

(54) SANTISING COMPOSITIONS AND METHODS

(75) Inventor: Dennis Keith Gilding, Wirral (GB)

(73) Assignees: Donna Gilding, Saint Louis, MO (US); Thomas Tuttle, San Francisco, CA (US)

(21) Appl. No.: 12/999,623

(22) PCT Filed: Jul. 22, 2009
(86) PCT No.: PCT/GB09/01817

§ 371 Date: Dec. 16, 2010

(30) Foreign Application Priority Data
Jul. 22, 2008 (GB) 0813404.1
Jul. 23, 2008 (GB) 0813473.6

(51) Int. Cl. A61K 8/43; A61K 31/155; A01N 37/52; A61P 31/02; A61Q 11/00; A01P 1/00

(52) U.S. Cl. 424/54; 514/635

(57) ABSTRACT

Sanitising compositions are provided for use in combination with cleaning compositions, cleaning and sanitization protocols with such compositions and applications of such sanitising compositions. The compositions comprise (a) biocidal component comprising one or more non-polymeric biguanides and analogs thereof, and one or more quaternary ammonium compounds and a formulation comprising (b) one or more cationic detergents having at least one unsubstituted alkyl group of 8 or more carbon atoms, (c) one or more chelating agents, and (d) an amine component comprising a mixture of at least one alkanolamine and at least one bis(aminoalkyl)alkylamine. The composition may be further enhanced by the incorporation of a mixture of surfactants comprises as a first component one or more non-ionic surfactants and as a second component one or more amphoteric surfactants. The composition is particularly effective when used in combination with a cleaning composition comprising the same mixture of surfactants.

### FIELD OF INVENTION

The present invention is concerned with sanitising compositions, their use in combination with cleaning compositions, cleaning and sanitisation protocols with such combinations and applications of such sanitising compositions.

### BACKGROUND ART

Infectious microorganisms are becoming an increasing problem around the world. For example, methicillin-resistant strains of Staphylococcus aureus (MRSA—also referred to as “golden staph”) are now a significant problem. Hospitals are examples of environments where MRSA acquired infections. These infections put the patient at risk, increase the cost of care, and reduce the number of beds available. In other environments such as dental rooms, laboratories, food preparation areas, schools etc, it is also important to prevent or minimise the growth of such bacteria, together with other microbial agents including fungi and viruses.

Hospitals and other environments requiring high levels of hygiene have a range of strategies to minimise the spread of such microorganisms. Prior to entering into surgery, patients are topically treated with antiseptic solutions. Carriers of MRSA are treated with such topical antiseptics, or in severe cases, are treated with antibiotics.

The known topical antiseptics cannot be relied upon to provide sufficient protection from such bacteria, especially as more resistant strains develop. They are also not capable of being applied in other presentation forms, such as oral tablets. Although surfaces in these environments are washed down with sterilising agents and disinfectants including bleaches containing chlorine in the form of hypochlorous acid/hypochlorite ion, these agents are quite corrosive, and other alternatives are desirable.

One particular phenomenon of importance in the spread of pathogens is the formation of biofilms. Biofilms are formed when micro-organisms adhere to a surface, where they grow and become a culture medium for more micro-organisms. A biofilm can be formed by a single species or micro-organism, for example a bacterium, fungus, algae, or protozoa. It is not uncommon for biofilms to be formed from multiple species of micro-organism; for example they may often be formed of multiple species of bacteria. In addition they may incorporate or be formed from debris which may be from living organisms, for example sebum or dead skin cells or alternatively or additionally the debris may be from inanimate sources, for example corrosion products.

Biofilm formation is a serious problem in the context of infection control, which may have a major impact in medical facilities where, inevitably, harmful pathogens such as Methicillin Resistant Staphylococcus aureus (MRSA), Legionella (responsible for legionnaires disease) and Escherichia coli (E. coli) may be present. Accordingly, there is a continued need for a means of combating pathogens in medical facilities and in particular there is a need to destroy, prevent the formation of, or alleviate the impact of, biofilms in hospital environments where the pathogens may flourish.

Sanitising agents containing alcohol and other biocidal components are commonly used to combat contamination of surfaces, such as human skin by pathogenic biological agents, such as bacteria, fungi and viruses. For generations, hand washing with soap and water has been considered a measure of personal hygiene. Then the concept of cleansing hands with an antiseptic agent emerged. Rinsing hands with an antiseptic agent was believed to be less effective than hand washing and was recommended only in emergencies or in areas where sinks were unavailable.

A major component of most of these antimicrobial compositions is alcohol (such as ethanol or isopropanol), which exhibits potent but transient antimicrobial effects based on physical disruption of cells and denaturation of key proteins. For this reason the majority of alcohol-based hand antiseptics contain either isopropanol, ethanol, n-propanol, or a combination of two of these products. Alcohol solutions containing 60%-95% alcohol are typically most effective, and higher concentrations are less potent because proteins are not denatured easily in the absence of water.

In addition to antimicrobial soaps and lotions, an additional class of antimicrobial agent is the alcohol-based sanitizer these are typically an alcohol-containing preparation designed for application to the hands for reducing the number of viable microorganisms on the hands. Such preparations typically contain 60%-95% ethanol or isopropanol.

However, while isopropyl alcohol has established antibacterial properties, it has the disadvantage that, when used regularly, it can cause skin irritation. As a result personnel may be reluctant to use such creams, soaps and other compositions comprising significant levels of isopropanol.

There are many antimicrobial or biocidal agents that are either used alone or in combination with other similar or supplementary agents in sanitising compositions to address some or all of these needs. All of these agents and compositions have their limitations and drawbacks. One major problem is the need to find sanitising compositions that have significant impact on a broad range of harmful pathogens (bacteria, fungi and viruses), whilst at the same time being safe to use in many applications. Typically the major biocidal components of any given sanitising composition are the most expensive component of the system. It is highly desirable therefore to obtain sanitising compositions that have good effects against pathogens utilizing lower levels of the major biocidal component(s) when compared to their normal use or where the effect of the major biocidal component(s) is enhanced. Another reason for attempting to keep the level of biocide as low as possible is to be able to provide compositions with higher levels of safety and low levels of side effects during use. This requirement is often competing with the need to increase the level of biocide in any given formulation to provide acceptable levels of efficacy.

Accordingly, it is an object of the present invention to provide new sanitising compositions, methods and preparations for sanitization of surfaces including hard surfaces, soft surfaces, human and animal skin and internal surfaces of the animal or human body.

### DETAILS OF THE INVENTION

The present invention is directed to a sanitising composition comprising:
(a) A biocidal component comprising one or more non-polymeric biguanides and analogs thereof of the structure (I),

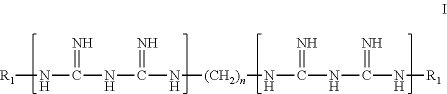

wherein n is 4, 6 or 8 and R₁ is a halogen substituted phenyl group, and one or more quaternary ammonium compounds of the structure (II),

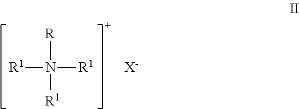

wherein R is a C₁₀ to C₂₀ unsubstituted branched or linear alkyl group and R₁ is a C₁ to C₄ branched or unbranched unsubstituted alkyl group and X is a halide ion,
(b) one or more cationic detergents having at least one unsubstituted alkyl group of 8 or more carbon atoms,
(c) one or more chelating agents, and
(d) an amine component comprising a mixture of at least one alkanolamine and at least one bis(aminoalkyl)alkylamine.

The first component of the biocide is one or more non-polymeric biguanides and analogs thereof of general structure (I). The halogen substitution may be chlorine or bromine, and is preferably chlorine. It is preferred that n is 6. The most preferred non-polymeric biguanides and analogs thereof of the structure (I) are chlorhexidine compounds in which n is 6 and R1 in structure (I) is a 4-chlorophenyl group. Chlorhexidine compounds include chlorhexidine free base as well as chlorhexidine salts, including but not limited to chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine succinamate, chlorhexidine malate, chlorhexidine tartrate, chlorhexidine dimonoglycolate, chlorhexidine mono-diglycolate, chlorhexidine dilactate, chlorhexidine di-.alpha.-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine isoethionate chlorhexidine di-2-hydroxy-napthoate, and chlorhexidine embonate. In the composition of the present invention it is preferred that the chlorhexidine is present as a salt. Preferred chlorhexidine salts include the acetates, formates, gluconates, hydrochlorides, isoethionates, lactates, and succinamates of chlorhexidine. The most preferred chlorhexidine salt is chlorhexidine di-gluconate.

The second component of the biocide is a quaternary ammonium compounds of the structure (II) in which organic radicals have been substituted for all four hydrogen's of the original ammonium cation. They have a central nitrogen atom, which is joined to four organic radicals. One of the organic radicals R in structure (II) is a C₁₀ to C₂₀ unsubstituted branched or linear alkyl group, the other three organic radicals denoted R₁ in structure (II) are a C₁ to C₄ branched or unbranched unsubstituted alkyl group. In a preferred antibiotic quaternary ammonium compound of structure (II) R is an alkyl group with 12 to 20 carbon atoms, preferably 12 to 18 carbon atoms and most preferably 12 to 16 carbon atoms. In a preferred embodiment the antibiotic quaternary ammonium compound of structure (II) comprises a mixture of two or more quaternary ammonium compounds in which the R group denoted in structure (II) has a different number of carbon atoms in each quaternary ammonium compound in the mixture. Preferably the mixture comprises three such quaternary ammonium compounds. In a preferred embodiment the three component mixture consists of a mixture of one quaternary ammonium compound having an R group as denoted in structure (II) of 12 carbon atoms, one quaternary ammonium compound having an R group as denoted in structure (II) of 14 carbon atoms and one quaternary ammonium compound having an R group as denoted in structure (II) of 16 carbon atoms. It is preferred that this mixture has a composition of 15-25% w/w C12, 75-85% w/w C14 and max 5% w/w C16. In the preferred antibiotic quaternary ammonium compounds of structure (II) the halide is bromide. The most preferred antibiotic quaternary ammonium compound of structure (II) is Cetrimide. Cetrimide is also known as alkyl trimethyl ammonium bromide. It is commonly understood to be a mixture of lauryltrimethylammonium bromide, myristyltrimethylammonium bromide, and palmityltrimethyl ammonium bromide, with composition 15-25% w/w C12, 75-85%w/w C14 and max 5% w/w C16.

In addition to the two part biocidal component the sanitising composition of the present invention has a further three components that in combination with the two component biocidal composition enhance the effectiveness of the biocidal composition. These three components are (a) one or more cationic detergents having at least one unsubstituted alkyl group of 8 or more carbon atoms, (b) one or more chelating agents, and (c) an amine component comprising a mixture of at least one alkanolamine with at least one bis(aminoalkyl)alkylamine.

The preferred cationic detergent is an alkyl dialkyl ammonium halide, preferably a chloride. Most preferably having at least one alkly group of 6 to 15 carbon atoms and more preferably 8 to 12 carbon atoms. Preferably the cationic detergent is an alkyl dimethylammonium halide and most preferably is didecyl dimethyl ammonium chloride. An example of a suitable commercial product is Bardac 22, manufactured and supplied by Lonza Ltd, Basel, Switzerland.

The chelating agent(s) useful in the sanitising compositions of the present invention are those selected from the group consisting of EDTA, disodium edetate,trans-1,2-diaminocyclohexane-N,N,N′,N′-tetraaceticacid monohydrate, N,N-bis (2-hydroxyethyl)glycine, 1,3-diamino-2-hydroxypropane-N,N,N′,N′-tetraacetic acid, 1,3-diaminopropane-N,N,N′,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N′,N′-triacetic acid, ethylenediamine-N,N,N′, N′-tetrakis(methylenephosponic acid), nitrilotriacetic acid, nitrilotripropionic acid, nitrilotris(methylenephosphoric acid), 7,19,30-trioxa-1,4,10,13,16,22,27,33-octaazabicyclo[11,11,11]pentatriacontane hexahydrobromide, triethylenetetramine-N,N,N′,N″,N′″,N′″-hexaacetic acid, pharmaceutically acceptable salts thereof, and mixtures thereof. The most preferred chelating agents are EDTA derivatives and most preferably the chelating agent is EDTA.

The amine component comprising a mixture of at least one alkanolamine with at least one bis(aminoalkyl)alkylamine further enhances the effectiveness of the biocidal component of the composition. The amines in the amine component may have biocidal activity. The alkanolamine may be a mono, di or tri-alkanolamine. It is preferred that the alkanolamine a monoalkanolamine with an alkyl substituent having from 2 to 6 carbon atoms, with the most preferred alkanolamine being monoethanolamine. The bis(aminoalkyl)alkylamine preferably comprises bis(aminoalkly) groups having from 1 to 12 carbon atoms, preferably 1 to 8 carbon atoms, more preferably 2 to 5 carbon atoms and most preferably are aminopropyl groups. The alkylamine group preferably has an alkyl group which is unsubstituted and may be branched or unbranced having from 3 to 24 carbon atoms, more preferably 6 to 20 carbon atoms, more preferably 6 to 18 carbon atoms, more preferably 8 to 16 carbon atoms and most preferably 8 to 12 carbon atoms. The preferred bis(aminoalkyl)alkylamines are bis(3-aminopropyl)decylamine and bis(3-aminopropyl)dodecylamine, with bis(3-aminopropyl)dodecylamine being the most preferred.

In a preferred embodiment of the present invention the sanitising composition further comprises a surfactant preferably one or more non-ionic surfactants. In a further embodiment the sanitising composition further comprises a mixture of surfactants, which enhance the effectiveness of the sanitising composition. In a preferred embodiment the mixture of surfactants comprises as a first component one or more non-ionic surfactants and as a second component one or more amphoteric surfactants.

Examples of nonionic surfactants include those selected from the group consisting of polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and mixtures thereof, alkyl polyglucosides, ceteth-2, ceteth-6, steareth-2, steareth-6, PEG-2 stearate, PPG-10 glyceryl stearate, and mixtures thereof. It is preferred that the nonionic surfactants used are surfactants, which are cleared for safe human contact and also preferably are biodegrable.

It is preferred that the non-ionic surfactant component of the mixture is selected from one or more alkyl polyglucosides, with alkyl groups containing 5 to 16 carbon atoms, preferably 5 to 14 carbon atoms, more preferably 6 to 12 carbon atoms, more preferably 8 to 12 carbon atoms and most preferably 10 to 12 carbon atoms in the hydrophobic alkyl group and with less than 12, preferably less than 10 and most preferably 8 or less glucose residues in the hydrophilic polyglucoside group. The most preferred alkyl polyglucosides are selected from the group consisting of lauryl polyglucoside, decyl polyglucoside and mixtures thereof.

In the composition the surfactant component may comprise from 0.1-45% by weight of the composition.

The one or more amphoteric components of the surfactant composition are preferably one or more amphoteric betaine surfactants. Typical alkyl dimethyl betaines include decyl betaine or 2-(N-decyl-N,N-dimethylammino)acetate, coco betaine or 2-(N-coc-N,N-dimethyl ammonio)acetate, myristyl betaine, palmityl betaine, lauryl betaine, cetyl betaine, cetyl betaine, stearyl betaine, etc. Examples of betaines also include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and amidosulfobetaines (wherein the RCONH(CH₂)₃ radical is attached to the nitrogen atom of the betaine) and oleyl betaine (available as amphoteric Velvetex OLB-50 from Henkel). A further class of amphoteric betaines are the alkylamidoalkylbetaines

It is preferred that the amphoteric betaine surfactants used in the surfactant component of the composition are one or more alkylamidoalkylbetaines. Preferably, the alkylamido group has from 6 to 20 carbon atoms, preferably 6 to 16 carbon atoms, more preferably 8 to 14 carbon atoms and most preferably 8 to 12 carbon atoms. Preferably the alkyl linking group has from 2 to 6 carbon atoms, more preferably from 2 to 4 carbon atoms and most preferably is 2 or 3 carbons atoms. The most preferred amphoteric betaines for use in the sanitising composition of the present invention are the cocoamidoalkyl betaines, namely cocoamidoethyl betaine and cocoamidopropyl betaine with cocoamidopropyl betaine being preferred and/or the lauramidoalkyl betaines with lauramidopropyl betaine being preferred. The betaines of choice are preferably the cocoamidopropyl betaine and, more preferably, the lauramido propyl betaine.

The sanitising compositions of the present invention may further comprise one or more of ethanol, isopropanol, and n-propanol. Although alcohol addiction is preferably avoided it has been found in some circumstances the addition of low levels of one or more of these alcohols from 1 to 10% by weight of the composition has a beneficial effect on the composition when used in eliminating spores such as clostridium difficile spores. The composition with alcohol additions at this level may be used at 40° C.

It is preferred that the sanitising composition of the present invention composition has a pH of 10 or less, preferably 9 or less and most preferably 8.5 or less. The compositions therefore preferably further comprise sufficient amounts of at least one pH modifier to ensure that the sanitising composition has a final pH of from about 3.0 to about 10.0, preferably from 4.0 to 9.0, and most preferably from 6.0 to 8.5. The pH modifiers useful in the present compositions include organic acids and organic bases and the like. Preferred non-limiting examples of pH modifiers useful to impart the desired pH to the present inventive compositions are those selected from the group consisting of acetic acid, succinic acid, malic acid, lactic acid, gluconic acid, tartaric acid, 1,2,3,4-butane tetracarboxylic acid, fumaric acid, sodium carbonate, sodium bicarbonate, and a mixture thereof. The preferred modifier is acetic acid.

The compositions of the present invention are free of chlorine bleach, caustics, acids, aldehydes, sulphonates, phosphates and borates. In addition the compositions and formulations do not have or produce malodours. The compositions of the present invention including any formulations incorporating the compositions are free of anionic additives such as for example anionic surfactants. The use of anionic additives may be disruptive to the micelle structure of the sanitising composition of the present invention.

In the sanitising compositions of the present invention it is preferred that the quaternary ammonium compound of structure (II), the cationic detergent, the bis(aminoalkyl)alkylamine and the nonionic surfactant when present are selected to have the major alkyl group of the same or similar number of carbon atoms. It is preferred therefore that these materials are selected to ensure that the major alkyl group has from 10 to 12 carbon atoms in that group. Selecting these major components to have such a commonality of carbon atoms in their major alkyl group has been found to be beneficial in providing a sanitising composition, which has a robust micelle structure.

In the sanitising compositions of the present invention preferred compositions in the following ranges of increasing preference moving left to right in as indicated in the following table. The balance to provide 100% being aqua with or without alcohol and small additions of buffer when required:

| | | | Most | Most |
|---|---|---|---|---|
| | | | Preferred | Preferred |
| Components | % w/w | % w/w | % w/w | % w/w |
| non-polymeric | 0.25-2 | 0.5-1.5 | 0.5-1.1 | |
| biguanides | | | | |
| quaternary ammonium | 0.25-2 | 0.5-1.5 | 0.5-1 | |
| compounds of the | | | | |
| structure (II) | | | | |
| cationic detergents | 2-15 | 3-12 | 5-12 | |
| having alkyl of 8 or | | | | |
| more carbon atoms | | | | |
| chelating agents | 0.1-5 | 0.1-4 | 0.1-3 | |
| alkanolamine | 0.25-8 | 0.25-6 | 0.5-5.5 | |
| bis(aminoalkyl)alkyl- | 0.25-10 | 0.25-9 | 0.3-8.5 | |
| amine | | | | |
| Nonionic Surfactant | 0.1-45 | 3-30 | 5-20 | 10-18 |
| Betaine | 1-25 | 3-20 | 5-18 | |

Surface sanitising compositions are compositions that are delivered to a surface to be sanitized. Such compositions typically being in the form of a liquid, an aerosol spray, or a volume of gel (such as a hydrogel) or lotion, and applied in sufficient quantity so as to substantially cover the surface to be sanitized with at least a thin film of the composition. Example surfaces that may be sanitized with such composition include hard surfaces, such as counters and tabletops, telephone handsets, and bathroom fixtures, along with soft surfaces, such as human skin. Accordingly, such compositions must be formulated so as to be compatible with such surfaces and their mode of use.

The sanitising compositions of the present invention may be used as such or may be incorporated into other formulations for various applications. Thus the sanitising compositions may be formulated as a sanitising composition in the form of: an aerosol; a hydrogel; a liquid composition; a lotion, preferably as a hand lotion; a hand wash; antibacterial wipes; a mouth wash; a shampoo; a body wash; a vaginal douche; a topical ointment; or nasal ointment; a surgical wash and/or irrigation solution, one preferred application is a cavity irrigation solution on operations involving access to the pericardium; a wash or coating composition for surgical instruments and or equipment; a wash or coating composition for surgical implants; as an additive for laundry processes and in compositions for use in animal husbandry.

Thus in an additional embodiment of the present invention, the sanitising compositions is used to formulate a composition in the form of a hand wash. Preferably the hand wash comprises from 2 to 10%, preferably 3 to 5% and most preferably 4% by weight of the sanitising composition according to the invention with 0.5 to 5 weight % emollient, preferably about 1 weight % emollient, with the balance being water. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof. A further example and preferred emollient is glycerol.

In an additional embodiment of the present invention, the sanitising composition according to the invention is formulated as a composition in the form of an antibacterial wipe in conjunction with a suitable wipe base material. A preferred wipe base is based on pulped cellulose. In a preferred embodiment the antibacterial wipe is an alcohol-free antibacterial wipe comprising: a flexible substrate preferably a fabric substrate (woven or non-woven) and a sanitising composition according to the invention.

In an additional embodiment of the present invention, the sanitising composition according to the invention is formulated as a composition in the form of a mouth wash. Preferably the mouth wash comprises from 2 to 10%, preferably 3 to 5% and most preferably 4% by weight of the sanitising composition according to the invention with 0.5 to 5 weight % emollient, preferably about 1% emollient, with 0.01 to 0.1 weight % flavouring, preferably 0.05 weight % mint flavouring the balance being water.

In an additional embodiment of the present invention, the sanitising composition according to the invention is formulated as a composition in the form of a shampoo. Preferably the shampoo comprises from 2 to 10%, preferably 3 to 5% and most preferably 4% by weight of the sanitising composition according to the invention, with 15% by weight of Surfac B4, 20% by weight Natrosol 250HHGF (hydroxy ethyl cellulose), 6% by weight of Cocoamide Diethanolamine, 15% by weight of Polysorbate T20, with the balance being water. In a further preferred formulation the shampoo comprises 4% by weight of the sanitising composition according to the invention, with 15% by weight of Surfac B4 betaine, 5% by weight quaternised Guar (AcquacatCG518), 6% by weight of Cocoamide MEA, 3% by weight of Methyl Gluceth, 4% by weight of PEG-120 Methyl Glucose Dioleate, with the balance being water.

In an additional embodiment of the present invention, the sanitising composition according to the invention is formulated as a composition in the form of a surgical wash and/or irrigation solution. Preferably the surgical wash and/or irrigation solution comprises from 2 to 10%, preferably 3 to 5% and most preferably 4% by weight of the sanitising composition according to the invention with 0.5 to 5 weight % emollient, preferably from 0.5 to 3% weight % emollient, and more preferably 1 to 3 weight % emollient and most preferably about 2 weight % emollient with the balance being water. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof. A further example and most preferred emollient is glycerol.

In further embodiments, the present invention provides a method of treating, alleviating or preventing microbial (bacterial, viral or fungal) disorders of the skin, body cavity or mucosal surface of a human or animal, wherein the disorder involves inflammation as one of its etiological factors, including administering topically to a subject having the disorder or which is in danger of developing the disorder, a sanitising composition according to the invention, wherein the antibiotic agents are administered in a therapeutically effective or preventative amount.

In one or more embodiments, the disorder to be treated is selected from the group consisting of a dermatose, a dermatitis, a vaginal disorder, a vulvar disorder, an anal disorder, a disorder of a body cavity, an ear disorder, a disorder of the nose, a disorder of the respiratory system, a bacterial infection, fungal infection, viral infection, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, pelvic inflammation, endometritis, salpingitis, oophoritis, genital cancer, cancer of the cervix, cancer of the vulva, cancer of the vagina, vaginal dryness, dyspare[upsilon]nia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

The present invention further provides a surface cleaning kit of parts, which comprises as a first component a degreaser/deep cleaning composition, which comprises a mixture of surfactants as hereinafter defined and described and as a second component a surface treatment/sanitizer composition comprising a the sanitising composition according to the invention with a mixture of surfactants as hereinbefore defined and described.

It is preferred that the non-ionic surfactant component of the degreaser/deep cleaning composition is a non-ionic surfactant, preferably a mixture two non-ionic surfactants. The first nonionic surfactant, which may be used alone, is selected from one or more alkyl polyglucosides, with alkyl groups containing 5 to 16 carbon atoms, preferably 5 to 14 carbon atoms, more preferably 6 to 12 carbon atom and most preferably 8 to 12 carbon atoms in the hydrophobic alkyl group and with less than 12, preferably less than 10 and most preferably 8 or less glucose residues in the hydrophilic polyglucoside group. The second non-ionic surfactant, which may be used in addition to the first in the non-ionic surfactant component is one or more alkyl ethoxylates, wherein the hydrophobic alkyl group has from 6 to 20 carbon atoms, preferably 6 to 16 carbon atoms and most preferably 8 to 12 carbon atoms and wherein the hydrophilic part has from 3 to 20 ethoxy residues, preferably from 3 to 16 ethoxy residues, more preferably from 3 to 12 ethoxy residues, more preferably from 3 to 8 ethoxy residues and most preferably from 3 to 6 ethoxy residues. The most preferred alkyl polyglucosides are selected from the group consisting of lauryl polyglucoside, decyl polyglucoside and mixtures thereof. In this surfactant component the ratio of glucoside to ethoxylate is preferably within the range of 0.8-10. The amount of alkyl polyglucoside is from 0.1-30% by weight on the surfactant component and the alkyl ethoxylates are from 0.05-15% by weight on the surfactant component. A further surfactant component present in the degreaser/deep cleaning composition may be an amphoteric betaine as used and described above in relation to the sanitising composition of the present invention.

In this embodiment the kit forms the basis of an effective cleaning regime to remove microbial contaminants from a surface especially a surface contaminated with a biofilm and to ensure that the surface remains microbe free for an extended period and ideally up to 3 days. The degreaser/deep cleaning composition is a surfactant-based composition with approximately 95% by weight of water and which comprises as the balance surfactants that have some commonality with the mixture as used in the formulation for the sanitising composition according to the invention as hereinbefore described. This commonality of surfactants between the two components of the kit results in the enhanced effectiveness of the kit in maintaining the cleaned surface microbe free for up to 3 days.

The basic kit comprising as a first component a degreaser/deep cleaning composition and as a second component a surface treatment/sanitizer may be used in a hospital cleaning protocol in the following fashion.

Firstly all surfaces require cleaning including: bed-frame and mattress; bedside table; light; chair(s); shelves and floor. The cleaning treatment begins with a ‘Deep Clean’ with the degreaser/deep cleaner, which is designed to lift and remove all forms of dead soil including the disruption of the biofilm that contains the microorganisms. During use surfactants in the composition condition all surfaces on which it is used by laying down a thin low energy hydrophobic layer that reduces the tendency to bind soil and when re-activated by water re-orients, becomes hydrophilic and enables freshly deposited soil to be washed away. Whereas high-energy surfaces such as glass and metal will adsorb and bind soil strongly, the surfactant coating reduces the tenacity of the binding of dirt and facilitates future removal.

Some of the surfactants used in the degreaser/deep cleaner are used in smaller concentration in the sanitising composition according to the invention, which has the same micelle structure as the degreaser/deep cleaner but in addition the micelles of the sanitising composition according to the invention when formulated with the surfactant mixture carry a cocktail of biocides and other agents. Since there is some identity of surfactants in both the deep cleaning and subsequent sanitising step this ensures that the two adsorbed sub layers are intimately bound together and act as an integral protective layer with soil repelling as well as antimicrobial properties.

The surfactant mixture containing the mixed non-ionic surfactants provides an exceptional ability to lift and remove organic contaminants far in excess of either component individually. Both are easily rinsed away, leaving no taint in the food industry. Each of the components is non-corrosive and biodegradable, either by simple hydrolysis or microbial digestion. The cleaning mixture is safe to use with minimal protective clothing. ‘In-line cleaning’ can be carried out without interrupting production because of hazards to personnel.

The kit has been assembled to enable a holistic approach to be taken for Infection Control in hospitals. The products are not only safe to use on and around people, but also in the environment in that they do not adversely affect water courses or sewage treatment systems. The aqueous antimicrobial composition or preparation of the present invention is Eco-friendly and when diluted below a critical threshold is degraded by the sewage treatment microorganisms into non-cumulative compounds within 30 days.

The first component (cleaner/degreaser) of the kit of the present invention, typically may have the following compositions based on parts by weight:

Formulation I

| Surfactant Component | |
|---|---|
| Alkyl polyglucoside (Plantacare 2000) | 195 |
| Alkyl ethoxylate (LT7 Ethoxylate) | 85 |
| Alkylamidoalkyl betaine (Betaine Surfac B4) | 95 |
| Formulation Additives | |
| GL 38 Glutamic acid | 15 |
| Sodium Carbonate | 10 |
| Pine Extract | 1 |
| Orange oil | 2 |
| Solvent Vehicle | |
| Isopropanol | 80 |
| Water | 419 |

Formulation II

| | |
|---|---|
| Surfactant Component | |
| Alkyl polyglucoside (Plantacare 2000) | 280 |
| Alkylamidoalkyl betaine (Betaine Surfac B4) | 95 |
| Formulation Additives | |
| GL 38 Glutamic acid | 15 |
| Sodium Carbonate | 10 |
| Pine Extract | 1 |
| Orange oil | 2 |
| Solvent Vehicle | |
| Isopropanol | 80 |
| Water | 419 |

The key components in this aspect of the present invention is that the non-ionic and amphoteric betaine surfactant combination being present in both the degreaser/deep cleaning composition and surface treatment/sanitiser composition comprising the sanitising composition according to the invention.

The system can be applied as a mist or spray, or with conventional mop and bucket. Tools used for sanitising is automatically sanitized during use and providing they are dedicated for use with the sanitising composition according to the invention. The sanitising composition according to the invention will retain an adsorbed anti microbial layer capable of lasting up to 3 days. The antimicrobial properties are enhanced and extended with each use.

Cleaning is carried out systematically within each work area from light soil to high soil. Care must be taken not to move soil from one work area to another to eliminate the vectors that cause recontamination particularly as part of barrier cleaning after a serious outbreak.

This total environmental cleaning/sanitising exercise conditions the hospital against future infections and is supplemented by single use specialty designed kits to eliminate specific vectoring during everyday procedures. These kits as described below provide various personal care items incorporating the sanitising composition according to the invention.

In a further embodiment the kit according to the present invention is optimized to provide all the components needed for effective infection control in a hospital environment. In this embodiment the second component is formulated into various formulations for application and use on contaminated or contaminatable surfaces and for treatment of the patient and or hospital staff and/or visitors to the hospital.

The kit may therefore further comprise in addition to the first component being a degreaser/deep cleaning composition, 1 tablet of Imperial Leather soap and one or more formulated products comprising the sanitising composition according to the invention and selected from one or more of the following: a surface sanitizer, a shampoo formulation, a body wash formulation, a mouth wash formulation, a hand sanitizer formulation and medicated wipes formulation. Preferably all of these components are present in the kit.

The patient (or family) is supplied with products to be able to use the sanitising composition according to the invention to cleanse and maintain the bed and immediate surroundings for a week.

The Imperial Leather soap is provided as a recent issue of ‘Which’ showed that this was more effective for sanitising hands than most proprietary hand cleansers, including alcohol gels without their drying effects on skin. Imperial Leather is used as a general washing agent.

The shampoo and body wash consist of 4% by weight of the sanitising composition according to the invention in a non-sulphate containing formulae as sulphates deactivate the aqueous antimicrobial composition or preparation.

The patient on entry should wipe down all surfaces immediately around the bed using the medicated Wipes. After doffing his/her outside clothes, he should shower or bath using the Imperial Leather soap as the primary cleansing system to flush away all background dirt from the outside. The shampoo and body wash should then be used to reduce the remaining microorganism count to a minimum. The hand wash is used for both as a sanitizer and as a cleansing solution for wounds and abrasions. Hand wash on a ‘Q’ Tip can be used to wipe around the nostrils to protect against MRSA, which tends to colonize the upper respiratory tract. The hand sanitizer solution protects 2-3 times more effectively than alcohol gel. This sanitizer contains an emollient to maintain the softness and elasticity of the skin. The hand lotion sanitizer deposits a protective coating to deliver more sanitising composition according to the invention than the liquid hand sanitizer. This layer sustains a higher level of the Infection Control. The emollient makes the skin in a soft, moist and elastic. After cleaning the teeth, the patient should use the mouthwash, even as a gargle, if there is any sign of a sore (infected) throat. The mouthwash is efficacious against cold sores and can be used on blisters around the mouth. Alternatively the mouth and nostrils can be wiped with a medicated wipe to obtain the same effect.

The patient's immediate surroundings should be cleaned daily after visitors. All visitors should use hand wash after washing their hands with soap and water.

In the event of a major infection outbreak in a hospital there is a need to cleanse and sanitize the unit(s) concerned including isolating the ward and implementing Barrier Cleaning. The following protocol should be used after an outbreak of infection:
(a) A single kit is provided for each bed in the infected unit.
(b) A trash-bag I set up for each contaminated bed for contaminated consumables, to be incinerated immediately after barrier cleaning.
(c) Cleaners should wash hands thoroughly with Imperial Leather soap and don aprons, gloves and overshoes.
(d) Mattresses are stripped of sheets/bedding as are curtains and all are placed in a soluble strip or fully soluble bag for laundering.
(e) Wash all surfaces in a deep cleaning cycle using the cleaner/degreaser to remove the heavy none live soil.
(f) Wash in a second cycle with sanitising composition system to ensure total decontamination.
(g) The ward should be dust-mopped with a static dry wipe on a mop, before being deep-cleaned using a dedicated mop with cleaner/degreaser and sanitized with sanitizer decomposition starting at the door and the moving through non-contaminated areas then through low contaminated areas to the heavily contaminated site. The use of formulations comprising the sanitising composition according to the invention automatically sanitizers the tools and they only need to be hung up until the next application.
(h) At no time is soil or wash fluid removed from the contaminated unit (bay) into other less contaminated areas this would be a major contaminating incident. Wash and spoil is immediately transferred and disposed of down the sluice.
(i) A combination of trigger spray and medicated wipes are used to clean and then sanitize the mattress, bed frame, light, bedside table, curtain rail, shelves and chair. Excess fluid is removed with paper towels. All used components are discarded immediately into the Trash-bag to prevent secondary contamination.
(j) Accidental heavy soiling of the gloves or apron or shoe-covers should be immediately discarded in the Trash-bag. The hands should be re-sanitized along with the replacement gloves and shoe covers with the hand sanitizer.
(k) The floor under the bed should be cleaned and sanitized with at trigger spray and squeegee and finished off with a wipe mop; the spoil being disposed of down the sluice, and the wipes in the trash. The mop and squeegee handles are cleaned and sanitized with a medicated wipe and stored as is.
(l) The apron, gloves and overshoes used around the contaminated bed should be changed prior to leaving the unit; the soles of the overshoes sprayed with the sanitising composition according to the invention. All contaminated items discarded in the Trash-bag.
(m) The sluice is the final area for deep cleaning with cleaner/degreaser and the sanitising composition according to the invention using medicated hand and mop wipes; special care to be taken in cleaning the commodes with strong wipes with an abrasive side to allow thorough scrubbing.
(n) All surfaces in the sluice are to be sprayed and wiped down with a series of the medicated wipes.

The Nidus of infection is usually a wound or orifice. The sanitising composition according to the invention has been formulated as a Wound and Orifice Cleanser i.e. Mouthwash without the flavouring. This ‘Biocide cocktail’ kills the local microorganisms, removes the source of infection and cleans the periphery. A variety of application methods are offered requiring a range of ‘User friendly’ product formats e.g. liquid, mist and foam sprays (to control delivery to horizontal and vertical surfaces).

Wipes with optimized texture (feel and applied friction) designed and selected to contain and control the application of biocides and most effectively remove both microorganisms and soil.

Body washes and shampoos containing the sanitising composition according to the invention will be offered for cleansing the patient's body and hair to lower overall micro organism count. After overall cleansing, the lotion acts as a water-soluble ‘barrier cream’ with extended anti-microbial properties to protect fragile skin around periphery. The same product will be used on nurse's hands to minimise re-contamination.

The sanitising composition according to the invention may be formulated with lower concentrations of surfactants and used as a laundry conditioner to provide an adsorbed residual anti-microbial activity for up to 24 hours for recyclable clothes e.g. gowns, nurses' uniforms, bed sheets, covers and drapes. These treatments are replenished during each laundry cycle.

In the case of disposable masks, caps, surgical drapes, patient gowns, sheets, nurses' and Carers' aprons and gloves, the sanitising composition according to the invention may be added as an anti-microbial treatment to the final stage of in-line fabric production by spraying or similar coating application procedure. Similarly, gloves fabricated by dipping are able to have adsorbable biocides incorporated into the final step to provide an anti-microbial outer surface. Incremental cost increase is minimal; added value is significant with greater anti-microbial protection to reduce recontamination. The various garments may be constructed from the anti-microbial treated fabric packaged and sterilized as usual, by radiation or Ethylene Oxide, but each disposable item will carry residual surface anti-microbial activity for up to 1-3 days in use.

The present invention provides a method of preventing or inhibiting growth of microorganisms. The compositions of the invention may be described as antibacterial or antimicrobial agents. The compositions may be described as disinfectants when formulated for application to surfaces or for dilution in aqueous solvents. The composition may be described as an antiseptic when formulated for application to a subject, including human or non-human animals. An antimicrobial agent is defined as a chemical compound (or preparation comprised of a mixture of two or more chemical compounds) capable of destroying or inhibiting the growth of microorganisms, such as bacteria, fungi, and viruses. A biocide is defined as chemical compound (or preparation comprised of a mixture of two or more chemical compounds) that is immediately destructive to many different microorganisms, typically due to physical disruption of such microorganisms. Accordingly, a bacteriocidal agent is a biocide that is immediately destructive to bacteria. A biostat is defined as a chemical compound (or preparation comprised of a mixture of two or more chemical compounds) that prevents or impedes proliferation of microorganisms, typically due to interference with a critical physiological pathway of such microorganisms. Accordingly, a bacteristatic agent is a biostat that prevents or impedes proliferation of bacteria. The sanitising compositions of the present invention and formulations based on these compositions may have one or more of these effects on microorganisms and in that sense are broad impact antimicrobial (impacting bacteria, fungi and viruses) compositions. The sanitising compositions of the present invention are effective disinfectants and may be termed as such.

In a further embodiment of the present invention there is provided a method of preventing or inhibiting growth of bacteria, the method comprising the step of contacting a surface with a formulation comprising the sanitising composition according to the invention. Preferably in the case of non-biological surfaces e.g. hard surfaces the method further comprises surface cleaning with a cleaner/degreaser according to the present invention. The formulation may be just the sanitising composition according to the invention, without any further dilution or additives.

Suitably the formulation comprising the composition(s) of the present invention is contacted with the microorganism by administering the composition(s) topically and/or orally to a subject in need thereof. Alternatively, the formulation may be applied to an inanimate surface comprising the microorganism or suspected or at risk of comprising the microorganism. The term “surface” is used in its broadest sense, and should not be read as implying any specific physical dimensions.

The formulation may be administered by any suitable route, and the person skilled in the art will readily be able to determine the most suitable route and dose for the condition to be treated. Dosage will be at the discretion of the attendant physician or veterinarian, and will depend on the nature and state of the condition to be treated, the age and general state of health of the subject to be treated, the route of administration, and any previous treatment which may have been administered.

The formulations may be administered to a subject in need thereof periodically or repeatedly, and may be administered to the site of actual or possible infection. For example, if infection of a surgical wound has occurred or is desired to be prevented, the formulations may be administered on or to the wound and around the wound. If infection of the nasal passages may occur or has occurred or is desired to be prevented, administration of the formulations to the nasal passages may be appropriate, and the formulations may be in the form of a nasal ointment or nasal spray. If infection of the throat has occurred or is desired to be prevented, the formulations may be in the form of a throat gargle, lozenge, or spray.

A suitable prophylactic treatment regime includes washing the patient with the formulations, optionally following a separate antiseptic treatment (depending on the components in the composition). This may be conducted on a periodic or repeating basis. The formulation may be in the form of a body wash, or otherwise a lotion may be applied followed by gauze. The formulation may be allowed to dry. The procedure may be repeated a number of times a day, with three times a day being suitable. The nasal ointment or spray may also be applied.

Preferred examples of bacteria treatable with the present antimicrobial compositions are gram positive bacteria, gram negative bacteria, and combinations thereof. Specific, non-limiting examples of such gram positive bacteria are those selected from the group consisting of Streptococcus sp., Micrococcus sp., Staphylococcus sp., Bacillus sp., and combinations thereof.

The compositions and methods of the present invention have been found to be particularly effective in relation to the following microorganisms, when tested under EN 1276, EN1040 and EN1275 testing conditions:
(a) MRSA—Six strains including EMRSA 15:CC22, EMRSA 16:CC30, IBERIAN CLONE:CC8, NEWYORK/TOKYO CLONE:CC5 and CA-MRSA USA 300 CLONE:CC8, SMRSA 153. These were netutarlised in under 1 minute of contact.
(b) C. diff (Vegatative), was neutralized in under 2 minutes of contact.
(c) C. diff (spores EN 13704), was neutralized in under 2 minutes of contact.
(d) EN1275 Candida Albicans, Aspergillus Niger.
(e) EN1276 Staph aureus, pseudomonas aeruginosa, E. coli, Ent. hirea.
(f) EN13697 2001 phase2/step2 surface test (as EN1276 in suspension).
(g) Salmonlella chloraesuis.
(h) Cinetobacter in less than 2 minutes contact.
(i) GRE (Vancomycin Resistant Enterococcus)
(j) SARS in less than five minutes contact.
(k) Influenza A
(l) Streptococcus Equus (Strangles).
(m) Listeria monocytogenes NCTC 11994.

The present method includes the sanitisation and treatment of surfaces and patients in contact with these microbes.

For the purposes of this application, the following definitions are used, and are believed to be consistent with conventional usage of such terms in the field.

For the avoidance of doubt, as used herein, the term “treatment” includes therapeutic and/or prophylactic treatment. As used herein, the singular forms “a”, “an”, and “the” include the corresponding plural reference unless the context clearly dictates otherwise. Thus, for example, a reference to “a virus” includes a plurality of viruses. A “microorganism” or “microbe” or “pathogen” is any organism of microscopic size. Microorganisms include bacteria, viruses, fungi such as yeasts and molds, algae, and the like.

“Disease” is a general term used herein to refer to any departure from health in which a subject suffers. A “condition” refers to an abnormal functioning of a function or part of a body.

The “subject” may be a mammal. The mammal may be a human, or may be a domestic or companion animal. While it is particularly contemplated that the compositions of the invention are suitable for use in humans, they are also applicable to veterinary use, including treatment of companion animals such as dogs and cats, and domestic animals such as horses, cattle and sheep, or zoo animals such as non-human primates, felids, canids, bovids, and ungulates.

The following are examples of a sanitising composition according to the invention:

### EXAMPLES 1 TO 5

The compositions of examples 1 to 5 as indicated in Table 1 were prepared by simple mixing and dissolution of the components in water.

TABLE 1

| | 1 | 2 | 3 | 4 | 5 |
|---|---|---|---|---|---|
| Component | weight | weight | weight | weight | weight |
| Alkyl Polyglucoside¹ | 32 | 28 | 28 | 28 | 28 |
| Betaine² | 28 | 32 | 32 | 12 | 12 |
| EDTA | 0.3 | 2 | 2 | 5 | 8 |
| Isopropanol | 3 | 0 | 0 | 0 | 0 |
| Bardac 22³ | 12 | 12 | 25 | 25 | 25 |
| Chlorhexidine Digluconate⁴ | 6 | 6 | 6 | 10 | 12 |
| Cetrimide | 2 | 2 | 2 | 2 | 2 |
| Monoethanolamine | 1.5 | 1.5 | 10 | 11 | 12 |
| Didecylmethylamine (Ex 1) | 1 | 1 | 10 | 15 | 20 |
| Bis(3 aminoproplyl)dodecylamine⁵ | | | | | |
| (other examples) | | | | | |
| Aqua | 120 | 120 | 120 | 120 | 120 |
| Pre-Buffer pH | 11 | 11.3 | 11.3 | 11.6 | 11.9 |
| pH after Acetic Acid buffering | 8.5 | 8.5 | 8.5 | 8.5 | 8.5 |

¹The alkyl polyglucoside was Decyl polyglucose Planataren 2000N as supplied by the Cognis Corporation.
²The betaine used was cocamidopropylbetaine.
³Bardac 22 as supplied by Lonza Group is an N,N-didecyl-N,N-dimethylammoniumchloride in isopropanol/water 26.5 to 30.5 weight % water and 19.5-24.5 weight % IPA. Approximately 51 weight % N,N-didecyl-N,N-dimethylammoniumchloride.
⁴20 weight/vol % in water.
⁵Lonzabac - 12.100 as supplied by Lonza Group. Maximum 1.5% water.

### EXAMPLE 6 AND 7

The composition of example 6 as indicated in Table 2 was prepared by simple mixing and dissolution of the components in water.

TABLE 2

| | Example 6 | Example 7 |
|---|---|---|
| Component | % w/w | % w/w |
| Decyl polyglucose (Plantaren 2000N)¹ | 14.6 | 13.6 |
| Cocamidopropyl betaine | 16.7 | 15.6 |
| Tetrasodium Glutamate Diacetate² | 0.2 | 0.2 |
| Bardac 22 | 3.2 | 2.98 |
| Chlorhexidine Digluconate | 0.6 | 0.5 |
| Cetrimide | 1.0 | 0.6 |
| Monoethanolamine | 0.8 | 0.73 |
| Bis(3 aminoproplyl)dodecylamine³ | 0.5 | 0.97 |
| Aqua | 62.4 | 64.82 |

¹As supplied by Cognis Corporation.
²As supplied by Akzo-Nobel Chelate GL-47
³As supplied by Lonza Group as Lonzabac 12.30 - approx 30 weight % amine, the balance being water.

### EXAMPLE 8

A sanitising composition of the present invention was evaluated under EN 1276.Chemical disinfectants and antiseptics—Quantitative suspension test for the evaluation of bactericidal activity of chemical disinfectants and antiseptics used in food, industrial, domestic and institutional areas (phase 2, step 1).

### Test Method and Its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile synthetic hard water |
| Product test concentrations | 80.0% W/V; 10.0% W/V; 5.0% V/V |
| Appearance product dilutions | Clear. |
| Contact time | t = 5 min ± 10 s |
| Test temperature | 20° C. ± 1° C. |
| Interfering substance | 3.0 g/l bovine albumin |
| Stability of mixture | No precipitation |
| Temperature of incubation | 37° C. ± 1° C. |
| Identification of strains. | Vancomycin resistant enterococcus |
| | NCTC 12201 |
| | Pseudomonas aeruginosa ATCC |
| | 15442 |
| | Escherichia coli ATCC 10536 |
| | Staphylococcus aureus ATCC 6538 |
| | Enterococcus hirae ATCC 8043 |

### Conclusion

According to testing carried out under conditions specified in EN 1276, the composition possesses bactericidal activity at a concentration of 5.0% V/V of the working concentration as tested after 5 minutes at 20° C. under clean conditions (0.3 g/L bovine serum albumin) for referenced strain Vancomycin resistant enterococcus NCTC 12201 and according to EN 1276, the composition possesses bactericidal activity at a concentration of 20.0% V/V of the working concentration as tested after 5 minutes at 20° C. under clean conditions (0.3 g/L bovine serum albumin) for referenced strains Pseudomonas aeruginosa ATCC 15442, Escherichia coli ATCC 10536, Staphylococcus aureus ATCC 6538 and Enterococcus hirae ATCC 8043

### EXAMPLE 9

A sanitising composition of the present invention was evaluated under EN1040. Chemical disinfectants and antiseptics—Basic bactericidal activity—Test method and requirements (phase 1).

### Test Method and Its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile synthetic hard water |
| Product test concentrations | 80.0% W/V; 50.0% W/V; 20.0% V/V |
| Appearance product dilutions | Clear. |
| Contact time | t = 5 min ± 10 s |
| Test temperature | 20° C. ± 1° C. |
| Interfering substance | 3.0 g/l bovine albumin |
| Stability of mixture | No precipitation |
| Temperature of incubation | 37° C. ± 1° C. |
| Identification of strains. | Pseudomonas aeruginosa ATCC |
| | 15442 |
| | Staphylococcus aureus ATCC 6538 |

### Conclusion.

According to EN 1040 (1997), the composition of the present invention at 20% V/V of the working concentration possesses bactericidal activity for the referenced strains Pseudomonas aeruginosa ATCC 15442 and Staphylococcus aureus ATCC 6538.

### EXAMPLE 10

A sanitising composition of the present invention was evaluated using a standard test method for efficacy of antimicrobial agents against viruses in suspension. HEPATITIS C VIRUS (BOVINE VIRAL DIARHOEA VIRUS SURROGATE)

### Test Method and Its Validation

| | |
|---|---|
| Method | Dilution -neutralization |
| Neutralizer: | Dulbecco's modified Eagles medium + 5% v/v horse |
| | serum |

### Experimental Conditions

| | |
|---|---|
| Product diluent used | Sterile synthetic hard water |
| Product test concentrations | 10.0% V/V; 20.0% V/V; 100.0% V/V |
| Appearance product dilutions | Clear. |
| Contact time | t = 5 min ± 10 s |
| Test temperature | 20° C. ± 1° C. |
| Interfering substance | 0.3 g/l bovine albumin |
| Stability of mixture | No precipitation |
| Temperature of incubation | 37° C. ± 1° C. |
| Identification of strains. | Bovine viral diarrhoea virus ATCC |
| | VR - 1422/BT cells |

### Conclusion.

The sanitisation composition of the present invention possesses virucidal activity in 5 minutes at 20° C. by showing a >4 log reduction in the viability of the Hepatitis C virus surrogate Bovine viral diarrhoea virus ATCC VR-1422.

### EXAMPLE 11

A sanitising composition of the present invention was evaluated using a standard test method for efficacy of antimicrobial agents against viruses in suspension. HERPES SIMPLEX VIRUS TYPE 1 (HUMAN HERPES VIRUS—1)

### Test Method and its Validation

| | |
|---|---|
| Method | Dilution -neutralization |
| Neutralizer: | Dulbecco's modified Eagles medium + 5% v/v fotal bovine |
| | serum |

### Experimental Conditions

| | |
|---|---|
| Product diluent used | Sterile synthetic hard water |
| Product test concentrations | 10.0% V/V; 20.0% V/V; 100.0% V/V |
| Appearance product dilutions | Clear. |
| Contact time | t = 5 min ± 10 s |
| Test temperature | 20° C. ± 1° C. |
| Interfering substance | 0.3 g/l bovine albumin |
| Stability of mixture | No precipitation |
| Temperature of incubation | 37° C. ± 1° C. |
| Identification of strains. | Human Herpes Virus -1 (ATCC VR- |
| | 733)/Vero cells |

### Conclusion.

The composition of the present invention as tested possesses virucidal activity in 5 minutes at 20° C. by showing a >4 log reduction in the viability of the Human Herpes Virus—1 (ATCC VR-733)/Vero cells

### EXAMPLE 12

A sanitising composition of the present invention was evaluated under EN 1276. STREPTOCOCCUS EQUI. and LISTERIA MONOCYTOGENES Chemical disinfectants and antiseptics—Quantitative suspension test for the evaluation of bactericidal activity of chemical disinfectants and antiseptics used in food, industrial, domestic and institutional areas (phase 2, step 1).

### Test Method and Its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile synthetic hard water |
| Product test concentrations | 100.0% V/V; 20.0% V/V; 10.0% V/V |
| Appearance product dilutions | Clear. |
| Contact time | t = 5 min ± 10 s |
| Test temperature | 20° C. ± 1° C. |
| Interfering substance | 3.0 g/l bovine albumin |
| Stability of mixture | No precipitation |
| Temperature of incubation | 37° C. ± 1° C. |
| Identification of strains. | Streptococcus equi (ATCC 33398) |
| | Listeria monocytogenes NCTC 11994 |

### Conclusion.

According to testing carried out under conditions specified in EN 1276, the sanitising composition of the present invention possesses bactericidal activity at a concentration of 10.0% V/V of the working concentration as tested after 5 minutes at 20° C. under clean conditions (0.3 g/L bovine serum albumin) for referenced strain Streptococcus equi ATCC 33398 and possesses bactericidal activity at a concentration of 10.0% V/V of the working concentration as tested after 5 minutes at 20° C. under clean conditions (0.3 g/L bovine serum albumin) for referenced strain Listeria monocytogenes NCTC 11994.

### EXAMPLE 13

A sanitising composition of the present invention was evaluated under EN 1276.COMMUNITY ACQUIRED METHICILLIN RESISTANT STAPHYLOCOCCUS AUREUS UK15 PVL+. Chemical disinfectants and antiseptics—Quantitative suspension test for the evaluation of bactericidal activity of chemical disinfectants and antiseptics used in food, industrial, domestic and institutional areas (phase 2, step 1).

### Test Method and its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile synthetic hard water |
| Product test concentrations | 100.0% V/V; 20.0% V/V; 10.0% V/V |
| Appearance product dilutions | Clear |
| Contact time | t = 5 min ± 10 s |
| Test temperature | 20° C. ± 1° C. |
| Interfering substance | 3.0 g/l bovine albumin |
| Stability of mixture | No precipitation |
| Temperature of incubation | 37° C. ± 1° C. |
| Identification of strains. | 03.8951.G - EMRSA 15 PVL+ |
| | VARIANT. |

### Conclusion.

According to testing carried out under conditions specified in EN 1276, the sanitising composition of the present invention possesses bactericidal activity at a concentration of 10.0% V/V of the working concentration as tested after 5 minutes at 20° C. under clean conditions (0.3 g/L bovine serum albumin) for referenced strain 03.8951.G—EMRSA 15 PVL+ VARIANT.

### EXAMPLE 14

A sanitising composition of the present invention was evaluated under EN 1276. COMMUNITY ACQUIRED METHICILLIN RESISTANT STAPHYLOCOCCUS AUREUS USA 300. Chemical disinfectants and antiseptics—Quantitative suspension test for the evaluation of bactericidal activity of chemical disinfectants and antiseptics used in food, industrial, domestic and institutional areas (phase 2, step 1).

### Test Method and its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile synthetic hard water |
| Product test concentrations | 100.0% V/V; 20.0% V/V; 10.0% V/V |
| Appearance product dilutions | Clear. |
| Contact time | t = 5 min ± 10 s |
| Test temperature | 20° C. ± 1° C. |
| Interfering substance | 3.0 g/l bovine albumin |
| Stability of mixture | No precipitation |
| Temperature of incubation | 37° C. ± 1° C. |
| Identification of strains. | 02.6225.E - SMRSA 153; MLST 8; |
| | USA 300. |

### Conclusion.

According to testing carried out under conditions specified in EN 1276, a sanitsing composition according to the present invention possesses bactericidal activity at a concentration of 10.0% V/V of the working concentration as tested after 5 minutes at 20° C. under clean conditions (0.3 g/L bovine serum albumin) for referenced strain 02.6225.E—SMRSA 153; MLST 8; USA 300.

### EXAMPLE 15

A sanitising composition of the present invention was evaluated under EN 1276.LEPTOSPIRA INTERROGANS Chemical disinfectants and antiseptics—Quantitative suspension test for the evaluation of bactericidal activity of chemical disinfectants and antiseptics used in food, industrial, domestic and institutional areas (phase 2, step 1).

### Test Method and its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile distilled water |
| Product test concentrations | 1.0% V/V; 2.0% V/V; 4.0% V/V |
| Appearance product dilutions | Clear. |
| Contact time | t = 5 min ± 10 s |
| Test temperature | 20° C. ± 1° C. |
| Interfering substance | 0.3 g/l bovine albumin |
| Stability of mixture | No precipitation |
| Temperature of incubation | 37° C. ± 1° C. |
| Identification of strains | Leptospira interrogans ATCC 23470 |

### Conclusion.

According to EN 1276, a sanitsing composition according to the present invention possesses bactericidal activity at a concentration of 4.00% V/V of the working concentration as tested after 5 minutes at 20° C. under CLEAN conditions (0,3 g/L bovine serum albumin) for referenced strains Leptospira interrogans ATCC 23470

### EXAMPLE 16

A sanitising composition of the present invention was evaluated under EN 1656. STREPTOCOCCUS UBERIS. Chemical disinfectants and antiseptics—Quantitative suspension test for the evaluation of bactericidal activity of chemical disinfectants and antiseptics used in veterinary field—Test method and requirements (phase 2/step 1)

### Test Method and its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile distilled water |
| Product test concentrations | 0.5% V/V; 1.0% V/V; 2.0% V/V |
| Appearance product dilutions | Clear. |
| Contact time | t = 30 min ± 10 s |
| Test temperature | 30° C. ± 1° C. |
| Interfering substance | 3.0 g/l bovine albumin |
| Stability of mixture | No precipitation |
| Temperature of incubation | 37° C. ± 1° C. |
| Identification of strains | Streptococcus uberis NCTC 8281 |

### Conclusion.

According to EN 1656, a sanitsing composition according to the present invention possesses bactericidal activity at a concentration of 0.5% V/V as tested after 30 minutes at 30° C. (TEAT DISINFECTANT MODIFICATION OF

EN 1656) under CLEAN conditions (3.0 g/L bovine serum albumin) for referenced strains Streptococcus uberis NCTC 8281.

### EXAMPLE 17

A sanitising composition of the present invention was evaluated under EN 1276. Klebsiella pneumoniae. Chemical disinfectants and antiseptics—Quantitative suspension test for the evaluation of bactericidal activity of chemical disinfectants and antiseptics used in food, industrial, domestic and institutional areas (phase 2, step 1).

### Test Method and Its Validation

| | |
|---|---|
| Method | Filtration-neutralization |
| | Neutralizer: Lecithin 3 g/l, Polysorbate |
| | 80 30 g/l, sodium thiosulphate 5 g/l, L- |
| | histidine 1 g/l, phosphate buffer |
| | 0.0025 mol/l, sterilized by autoclave. |

### Experimental Conditions

| | |
|---|---|
| Product diluent used | Sterile synthetic hard water |
| Product test concentrations | 100.0% V/V; 20.0% V/V; 10.0% V/V |
| Appearance product dilutions | Clear. |
| Contact time | t = 5 min ± 10 s |
| Test temperature | 20° C. ± 1° C. |
| Interfering substance | 3.0 g/l bovine albumin |
| Stability of mixture | No precipitation |
| Temperature of incubation | 37° C. ± 1° C. |
| Identification of strains. | Klebsiella pneumoniae NCIB 10341 |

### Conclusion.

According to testing carried out under conditions specified in EN 1276, a sanitsing composition according to the present invention possesses bactericidal activity at a concentration of 20.0% V/V of the working concentration (which was diluted from a concentrate and adjusted ×1.25) as tested after 5 minutes at 20° C. under clean conditions (0.3 g/L bovine serum albumin) for referenced strain Klebsiella pneumoniae NCIB 10341.

### EXAMPLE 18

A sanitising composition of the present invention was evaluated under a standard test method for efficacy of antimicrobial agents against viruses in suspension. INFLUENZA A VIRUS (H1N1)

### Test Method and its Validation

| | |
|---|---|
| Method | Dilution -neutralization |
| Neutralizer: | Dulbecco's modified Eagles medium + |
| | 5% v/v foetal bovine serum. Virus |
| | detection by haemagglutination assay. |

### Experimental Conditions

| | |
|---|---|
| Product diluent used | Sterile synthetic hard water |
| Product test concentrations | 1.0% V/V; 2.0% V/V; 4.0% V/V |
| Appearance product dilutions | Clear. |
| Contact time | t = 5 min ± 10 s |
| Test temperature | 20° C. ± 1° C. |
| Interfering substance | 0.3 g/l bovine albumin |
| Stability of mixture | No precipitation |
| Temperature of incubation | 37° C. ± 1° C. |
| Identification of strains. | Influenza A (H1N1) (TC Adapted) |
| | (ATCC- VR-1469)/MDCK cells |

### Conclusion.

A sanitsing composition according to the present invention as tested possesses virucidal activity in 5 minutes at 20° C. by showing a >2.5 log reduction in the viability of the Influenza A (H1N1) (TC Adapted) (ATCC-VR-1469)/MDCK cells.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

In the specification, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A sanitising composition comprising:
(a) A biocidal component comprising:
(i) a one or more non-polymeric biguanides, salts or analogs thereof, of structure (I), 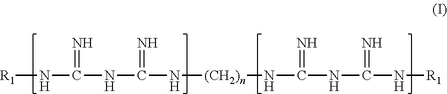
wherein n is 4, 6 or 8 and R₁ is a halogen substituted phenyl group; and
(ii) one or more quaternary ammonium compounds of structure (II), 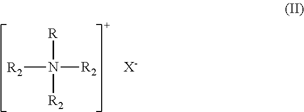
wherein R is a C₁₀ to C₂₀ unsubstituted branched or linear alkyl group and R₂ is a C₁ to C₄ branched or unbranched unsubstituted alkyl group and X is a halide ion,
(b) one or more cationic detergents having at least one unsubstituted alkyl group of 8 or more carbon atoms,
(c) one or more chelating agents, and
(d) an amine component comprising at least one alkanolamine and at least one bis(aminoalkyl)alkylamine.

2. The sanitising composition as claimed in claim 1 wherein in structure (I) n is 6 and R₁ is a 3-chlorophenyl group.

3. The sanitising composition as claimed in claim 1 wherein the non-polymeric biguanides is chlorhexidine di-gluconate.

4. The sanitising composition as claimed in claim 1 wherein the cationic detergent is didecyl dimethyl ammonium chloride.

5. The sanitising composition of claim 1 wherein the composition has a pH of 10 or less.

6. The sanitising composition as claimed in claim 1 wherein the chelating agent is EDTA.

7. The sanitising composition as claimed in claim 1 wherein each R of the one or more quaternary ammonium compounds of structure (II) has 12 to 20 carbon atoms.

8. The sanitising composition as claimed in claim 7 wherein each R of the one or more quaternary ammonium compounds of the structure (II) has a different numbers of carbon atoms.

9. The sanitising composition as claimed in claim 8 wherein the one or more quaternary ammonium compounds of the structure (II) include a mixture of three quaternary ammonium compounds.

10. The sanitising composition as claimed in claim 9 wherein the mixture comprises lauryltrimethylammonium bromide, myristyltrimethylammonium bromide, and palmityltrimethylammonium bromide.

11. The sanitising composition as claimed in claim 9 wherein the mixture comprises 15-25% w/w C12, 75-85%w/w C14 and max 5% w/w C16.

12. The sanitising composition as claimed in claim 11 wherein the quaternary ammonium compounds of the structure (II) is Cetrimide.

13. The sanitising composition as claimed in claim 1 wherein the bis(aminoalkyl) component has alkyl groups of 1 to 12 carbon atoms, and the alkylamine component has 3 to 24 carbon atoms.

14. The sanitising composition as claimed in claim 13 wherein the bis(aminoalkyl)alkylamine is bis(3-aminopropyl)dodecylamine.

15. The sanitising composition as claimed in claim 1 wherein the alkanolamine comprises one or more monoalkanolamines.

16. The sanitising composition as claimed in claim 1 further comprising one or more of ethanol, isopropanol, and n-propanol.

17. The sanitising composition as claimed in claim 1 further comprising a mixture of surfactants comprises as a first component one or more non-ionic surfactants and as a second component one or more amphoteric surfactants.

18. The sanitising composition as claimed in claim 17 wherein the amphoteric surfactant is one or more amphoteric betaine surfactants.

19. The sanitising composition as claimed in claim 18 wherein the betaine is an alkylamidoalkylbetaine wherein the alkylamido group contains 8-12 carbon atoms.

20. The sanitising composition as claimed in claim 19 wherein the alkylamidoalkylbetaine is cocoamidopropyl betaine.

21. The sanitising composition as claimed in claim 17 wherein the non-ionic surfactant comprises one or more alkyl polyglucosides with alkyl groups containing 8 to 12 carbon atoms and the polyglucoside (hydrophilic) has less than 8 glucose residues.

22. The sanitising composition of claim 1 in the form of an aerosol.

23. The sanitising composition of claim 1 in the form of a hydrogel.

24. The sanitising composition as claimed in claim 1 in the form of a hand lotion.

25. The sanitising composition as claimed in claim 1 in the form of a hand wash.

26. The sanitizing composition as claimed in claim 1 in the form of a medicated wipe.

27. The sanitising composition as claimed claim 1 in the form of a liquid.

28. The sanitising composition as claimed in claim 1 in the form of a mouthwash.

29. The sanitising composition as claimed in claim 1 in the form of a shampoo.

30. The sanitising composition as claimed claim 1 in the form of a body wash.

31. The sanitising composition as claimed claim 1 in the form of a vaginal douche.

32. The sanitising composition as claimed in claim 1 in the form of a topical ointment or a nasal ointment.

33. The sanitising composition as claimed in claim 1 in the form of a surgical wash and/or irrigation solution.

34. The sanitising composition as claimed in claim 33 further comprising an emollient.

35. The sanitising composition as claimed in claim 1 in the form of a wash or coating composition for surgical instruments and or equipment.

36. The sanitising composition as claimed in claim 1 in the form of a wash or coating composition for surgical implants.

37. The sanitising composition as claimed in claim 1 in the form as an additive for laundry processes.

38. The sanitising composition as claimed in claim 1 in the form for use in animal husbandry.

39. The sanitising composition as claimed in claim 1 in the form for use in the treatment of food products.

40. A method of treating, alleviating or preventing microbial (bacterial, viral or fungal) disorders of the skin, body cavity or mucosal surface of a human or animal, comprising: administering topically to the human or animal having the disorder or in danger of developing the disorder, a medicament comprising a sanitising composition having:
(a) A biocidal component comprising:
(i) one or more non-polymeric biguanides, salts or analogs thereof, of structure (I), 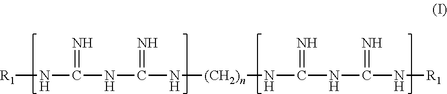
wherein n is 4, 6 or 8 and R₁ is a halogen substituted phenyl group; and
(ii) one or more quaternary ammonium compounds of structure (II), 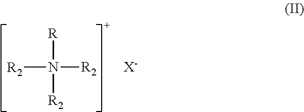
wherein R is a C₁₀ to C₂₀ unsubstituted branched or linear alkyl group and R₂ is a C₁ to C₄ branched or unbranched unsubstituted alkyl group and X is a halide ion,
(b) one or more cationic detergents having at least one unsubstituted alkyl group of 8 or more carbon atoms,
(c) one or more chelating agents, and
(d) an amine component comprising at least one alkanolamine and at least one bis(aminoalkyl)alkylamine.
wherein the biocidal components are administered in a therapeutically effective or preventative amount.

41. A surface cleaning kit comprising:
a first component including a degreaser/deep cleaning composition comprising a mixture of surfactants; and
a second component including a sanitising composition comprising:
(a) A biocidal component comprising:
(i) one or more non-polymeric biguanides, salts or analogs thereof, of structure (I), 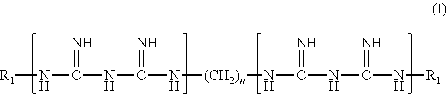
wherein n is 4, 6 or 8 and R₁ is a halogen substituted phenyl group; and
(ii) one or more quaternary ammonium compounds of structure (II), 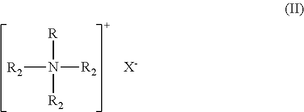
wherein R is a C₁₀ to C₂₀ unsubstituted branched or linear alkyl group and R₂ is a C₁ to C₄ branched or unbranched unsubstituted alkyl group and X is a halide ion,
(b) one or more cationic detergents having at least one unsubstituted alkyl group of 8 or more carbon atoms,
(c) one or more chelating agents,
(d) an amine component comprising at least one alkanolamine and at least one bis(aminoalkyl)alkylamine; and
(e) a mixture of surfactants comprises as a first component one or more non-ionic surfactants and as a second component one or more amphoteric betaine surfactants.

42. The surface cleaning kit of claim 41 wherein the degreaser/deep cleaning composition comprises the same surfactant mixture as those in the sanitising composition.

43. A cleaning and sanitization kit comprising two or more formulated products, at least one of the formulated products comprising:
(a) A biocidal component comprising:
(i) one or more non-polymeric biguanides, salts or analogs thereof, of structure (I), 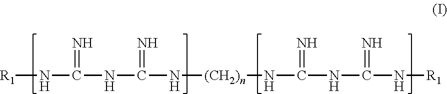
wherein n is 4, 6 or 8 and R₁ is a halogen substituted phenyl group; and
(ii) one or more quaternary ammonium compounds of structure (II), 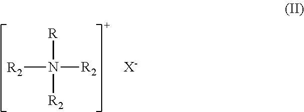
wherein R is a C₁₀ to C₂₀ unsubstituted branched or linear alkyl group and R₂ is a C₁ to C₄ branched or unbranched unsubstituted alkyl group and X is a halide ion,
(b) one or more cationic detergents having at least one unsubstituted alkyl group of 8 or more carbon atoms,
(c) one or more chelating agents, and
(d) an amine component comprising at least one alkanolamine and at least one bis(aminoalkyl)alkylamine.

44. A cleaning and sanitization kit as claimed in claim 43 which comprises 1 tablet of Imperial Leather soap and one or more formulated products selected from: a surface sanitizer, a shampoo formulation, a body wash formulation, a mouth wash formulation, a hand sanitizer formulation and medicated wipes formulation.

45. A method of cleaning a microorganism-contaminated surface or preventing or inhibiting growth of a microorganism on a surface, comprising contacting the surface with a sanitising composition comprising:
(a) A biocidal component comprising:
(i) one or more non-polymeric biguanides, salts or analogs thereof of, structure (I), 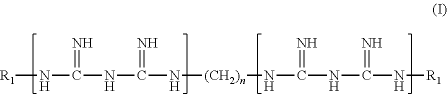
wherein n is 4, 6 or 8 and R₁ is a halogen substituted phenyl group; and
(ii) one or more quaternary ammonium compounds of the structure (II), 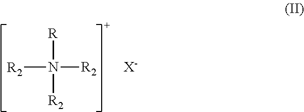
wherein R is a C₁₀ to C₂₀ unsubstituted branched or linear alkyl group and R₂ is a C₁ to C₄ branched or unbranched unsubstituted alkyl group and X is a halide ion,
(b) one or more cationic detergents having at least one unsubstituted alkyl group of 8 or more carbon atoms,
(c) one or more chelating agents, and
(d) an amine component comprising at least one alkanolamine and at least one bis(aminoalkyl)alkylamine.

46. The method of claim 45, further comprising: cleaning the surface with a deep cleaner/detergent having nonionic surfactants prior to sanitising the surface with the sanitising composition wherein the sanitising composition comprises the same nonionic surfactants as the deep cleaner/detergent.
